(19) United States
(12) Patent Application Publication
(10) Pub. No.: US 2022/0031139 A1
(43) Pub. Date: Feb. 3, 2022

(54) Mobile disinfection cart

(71) Applicant: YUN-YUEH CHEN, Taichung City (TW)

(72) Inventor: YUN-YUEH CHEN, Taichung City (TW)

(21) Appl. No.: 16/942,792

(22) Filed: Jul. 30, 2020

(30) Foreign Application Priority Data
Jul. 29, 2020 (TW) 109125528

(51) Int. Cl. A47L 11/40; B60K 1/00; A61L 9/14; A47L 11/293; B01D 46/00

(52) U.S. Cl. CPC A47L 11/4027; B60K 1/00; A61L 9/14; A61L 2101/06; A47L 11/4038; B01D 46/0028; A47L 11/293

(57) ABSTRACT

A mobile disinfection cart contains: a body, a drive system, a water absorption rake, a brush disc, a lid, an air inlet defined on a rear end of the lid, a sterilization structure accommodated in the cover, located above the chassis, facing the drive system, and communicating with the air inlet, an air outlet defined on the lid, an air conduit communicating with the sterilization structure and the air outlet. The mobile disinfection cart further contains a filter fixed in an air conduit, a storage tank secured on the body and communicating with the brush disc, an accommodation box disposed on the body and being in communication with the sterilization structure, and a power supply fixed on the body and electrically connected with the drive system and the sterilization structure.

### FIELD OF THE INVENTION

The present invention relates to cleaning equipment, and more particularly to a mobile disinfection cart which is capable of washing ground/floor, providing cleaned airs, and sterilizing.

### BACKGROUND OF THE INVENTION

A conventional floor scrubber is applied to clean ground/floor and to absorb foul water, and the conventional washing machine is in types of semi-automatic floor scrubber, automatic floor scrubber, hand-push scrubber, driving scrubber.

However, the conventional floor scrubber cannot sterilize and disinfect to virus, such as COVID-19, SARS and Influenza.

The present invention has arisen to mitigate and/or obviate the afore-described disadvantages.

### SUMMARY OF THE INVENTION

The primary aspect of the present invention is to provide a mobile disinfection cart which is capable of washing ground/floor, providing cleaned airs, and sterilizing.

To obtain the above-mentioned aspects, a mobile disinfection cart provided by the present invention contains: a body, a drive system, a water absorption rake, a brush disc, a lid, an air inlet, a sterilization structure, an air outlet, an air conduit, a filter, a storage tank, an accommodation box, and a power supply.

The drive system is disposed on a front end of the body.

The water absorption rake is fixed on a rear end of the body.

The brush disc is mounted on a bottom of the body.

The lid is arranged on a top of the rear end of the body.

The air inlet is defined on a rear end of the lid and located above the rear end of the body.

The sterilization structure is accommodated in the cover, is located above the rear end of the chassis, faces the drive system, and communicates with the air inlet.

The air outlet is defined on a top of the lid.

The air conduit communicates with the sterilization structure and the air outlet.

The filter is fixed in an air conduit and is located above the sterilization structure, and the air outlet is located above the filter.

The storage tank is secured on the body and is in communication with the brush disc.

The accommodation box is disposed on the body and is in communication with the sterilization structure.

The power supply is fixed on the body and is electrically connected with the drive system and the sterilization structure.

The sterilization structure includes a motor, a fan, a disinfecting tube, a main controller configured to control the motor and the fan, a heater controller, a delivery pipe configured to deliver chlorine dioxide, a control valve, a heating ring, a fixer configured to fix the heating ring, a stop mesh element, an opening, and a rotatable spray ring.

The motor is electrically connected with the power supply, a front end of the fan is connected with the motor, and a rear end of the fan is coupled with the disinfecting tube. The main controller and the heater controller are mounted on an outer wall of the disinfecting tube and are electrically connected with the power supply. The delivery pipe is in communication with the accommodation box and the disinfecting tube, the control valve is fixed on a predetermined position of the delivery pipe and is controlled by the heater controller, and the fixer is secured on an end of the disinfecting tube away from the fan, the heating ring is disposed on an end of the fixer away from the disinfecting tube. The stop mesh element is mounted on an outer wall of an end of the heating ring away from the fixer, the central opening is defined on the end of the heating ring away from the fixer, and the central opening has a through hole formed on an inner wall thereof. The rotatable spray ring is fixed on an end of the central opening away from the heating ring.

Preferably, the filter is a High-Efficiency Particulate Air (HEPA) filter.

Preferably, the stop mesh element has 400 meshes.

Preferably, the rotatable spray ring has a coupling tube and multiple aerosol nozzles, the coupling tube passes through the through hole of the central opening and communicates with the accommodation box, and the multiple aerosol nozzles are spaced angularly, face inward, and communicate with the coupling tube.

Preferably, the storage tank and the accommodation box are defined between the drive system and the water absorption rake.

Preferably, the storage tank includes a detachable cap, and the accommodation box includes a removable cover.

Preferably, the chassis has a connection tab connected on the rear end of the lid by a rotary shaft so as to uncover and cover the lid.

### BRIEF DESCRIPTION OF THE DRAWINGS

FIG. 1 is a perspective view showing the assembly of a mobile disinfection cart according to a preferred embodiment of the present invention.

FIG. 2 is a perspective view showing the assembly of a part of the mobile disinfection cart according to the preferred embodiment of the present invention.

FIG. 3 is a side plan view showing a part of the assembly of the mobile disinfection cart according to the preferred embodiment of the present invention.

FIG. 4 is a side plan view showing the operation of the mobile disinfection cart according to the preferred embodiment of the present invention.

FIG. 5 is another side plan view showing the operation of the mobile disinfection cart according to the preferred embodiment of the present invention.

### DETAILED DESCRIPTION OF THE FIRST EMBODIMENTS

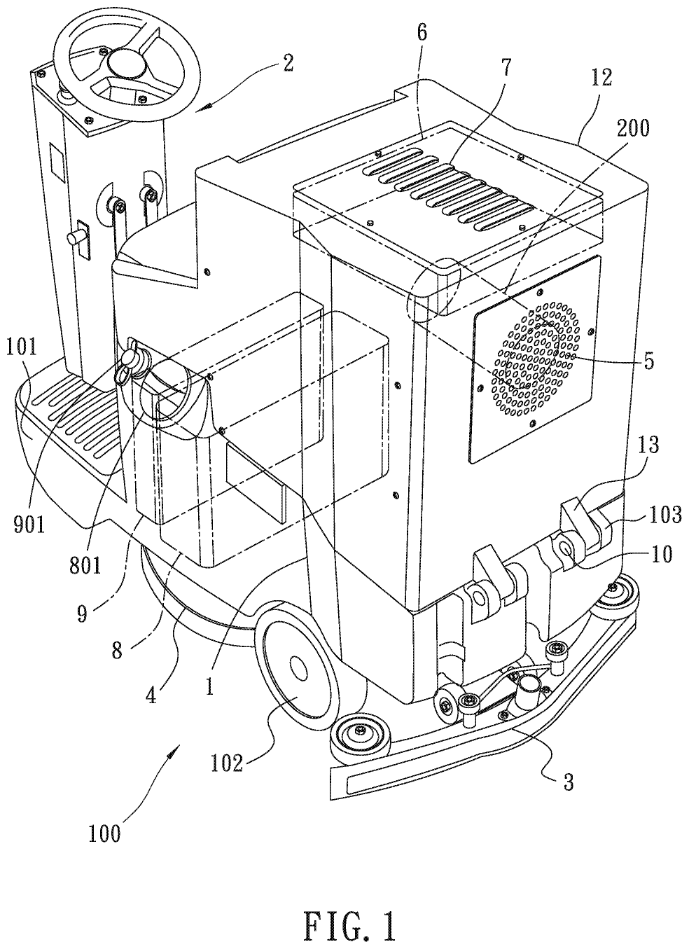

With reference to FIG. 1, a mobile disinfection cart 100 according to a preferred embodiment of the present invention comprises: a body 1, a drive system 2, a water absorption rake 3, a brush disc 4, an air inlet 5, a sterilization structure 200, a filter 6, an air outlet 7, a storage tank 8, an accommodation box 9, and a power supply P.

Referring to FIG. 1, the body 1 includes a chassis 101 and a wheel set 102 (only a wheel of the wheel set is shown).

The drive system 2 is disposed on a front end of the body 1 (i.e. the chassis 101) and is electrically connected with the power supply P so as to control the body 1 to move.

The water absorption rake 3 is fixed on a rear end of the body 1 (i.e. the chassis 101).

The brush disc 4 is mounted on a bottom of the body 1 (i.e. the chassis 101).

The storage tank 8 is secured on the body 1 (i.e. the chassis 101) and is in communication with the brush disc 4, such that after the drive system 2 is operated by a user to control the body 1 to move, the storage tank 8 supplies waters to the brush disc 4 so that the brush disc 4 cleans and washes a ground/floor, and the water absorption rake 3 scraps and absorbs the waters on the ground/floor.

In another embodiment, the body 100 further includes a lid 12 arranged on a top of the rear end of the chassis 101 and configured to cover and uncover the chassis 101, thus replacing or maintaining the sterilization structure.

The air inlet 5 is defined on a rear end of the lid 12 and is located above the water absorption rake 3.

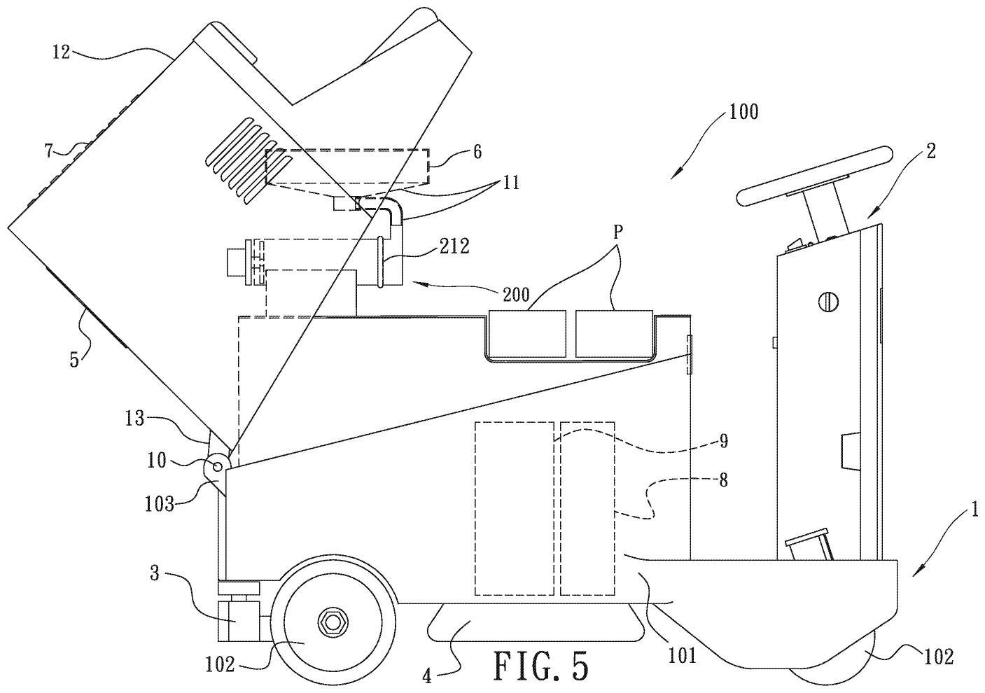

In another embodiment, as shown in FIG. 5, the chassis 101 has a connection tab 103 connected on the rear end of the lid 12 by a rotary shaft 10 so as to uncover and cover the lid 12.

The sterilization structure 200 is accommodated in the cover 12, is located above the rear end of the chassis 101, faces the drive system 2, and communicates with the air inlet 5.

The filter 6 is fixed in an air conduit 11 and is located above the sterilization structure 200. In another embodiment, the filter 6 is a High-Efficiency Particulate Air (HEPA) filter.

The air outlet 7 is defined on a top of the lid 12 and is located above the filter 6.

The accommodation box 9 is disposed on the body 1 (i.e. the chassis 101) and is in communication with the sterilization structure 200. In another embodiment, the storage tank 8 and the accommodation box 9 are defined between the drive system 2 and the water absorption rake 3. The storage tank 8 includes a detachable cap 801, and the accommodation box 9 includes a removable cover 901. Thereby, the detachable cap 801 is opened so as to supplement the waters into the storage tank 8 after running out of the waters and to cover the storage tank 8 after feeding the new waters. After running out carbon dioxide, the detachable cap 801 is opened so as to supplement the carbon dioxide into the accommodation box 9, and then the removable cover 901 is covered to the accommodation box 9.

The power supply P is fixed on the body 1 (i.e. the chassis 101) and is electrically connected with the drive system 2 and the sterilization structure 200.

The body 100 includes the air conduit 11 which communicates with the sterilization structure 200 and the air outlet 7 so as to guide clean airs out of the air outlet 7 via the air conduit 11, thus cleaning airs.

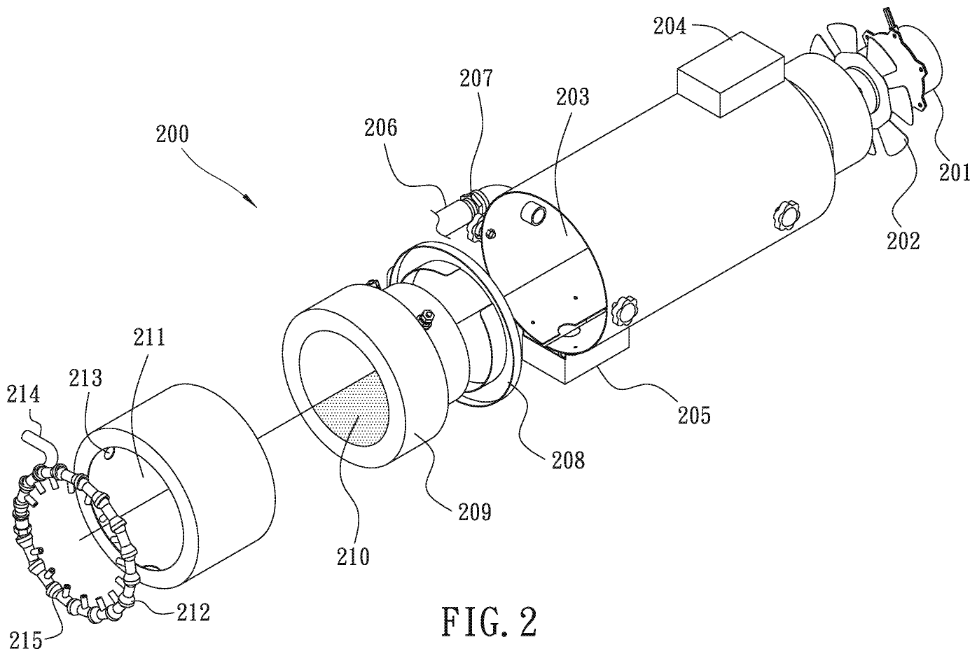

As shown in FIG. 2, the sterilization structure 200 includes a motor 201, a fan 202, a disinfecting tube 203, a main controller 204 configured to control the motor 201 and the fan 202, a heater controller 205, a delivery pipe 206 configured to deliver chlorine dioxide, a control valve 207, a heating ring 209, a fixer 208 configured to fix the heating ring 209, a stop mesh element 210, an opening 211, and a rotatable spray ring 212.

The motor 201 is electrically connected with the power supply P. A front end of the fan 202 is connected with the motor 201, and a rear end of the fan 202 is coupled with the disinfecting tube 203. The main controller 204 and the heater controller 205 are mounted on an outer wall of the disinfecting tube 203 and are electrically connected with the power supply P. In another embodiment, as illustrated in FIG. 2, the main controller 204 is mounted on the outer wall of the disinfecting tube 203 opposite to the heater controller 205. The delivery pipe 206 is in communication with the accommodation box 9 and the disinfecting tube 203. The control valve 207 is fixed on a predetermined position of the delivery pipe 206 and is controlled by the heater controller 205 so as to control the carbon dioxide to flow into the disinfecting tube 203. The fixer 208 is secured on an end of the disinfecting tube 203 away from the fan 202, the heating ring 209 is disposed on an end of the fixer 208 away from the disinfecting tube 203, and the stop mesh element 210 is mounted on an outer wall of an end of the heating ring 209 away from the fixer 208. In another embodiment, the stop mesh element 210 has 400 meshes. The central opening 211 is defined on the end of the heating ring 209 away from the fixer 208, and the central opening 211 has a through hole 213 formed on an inner wall thereof. The rotatable spray ring 212 is fixed on an end of the central opening 211 away from the heating ring 209.

With reference to FIG. 2, the rotatable spray ring 212 has a coupling tube 214 and multiple aerosol nozzles 215. The coupling tube 214 passes through the through hole 213 of the central opening 211 and communicates with the accommodation box 9, and the multiple aerosol nozzles 215 are spaced angularly, face inward, and communicate with the coupling tube 214. The heater controller 205 is turned on or off to control the heating ring 209 to heat up to 100° C., and the heater controller 205 also controls the control valve 207 so that the multiple aerosol nozzles 215 spray aerosol.

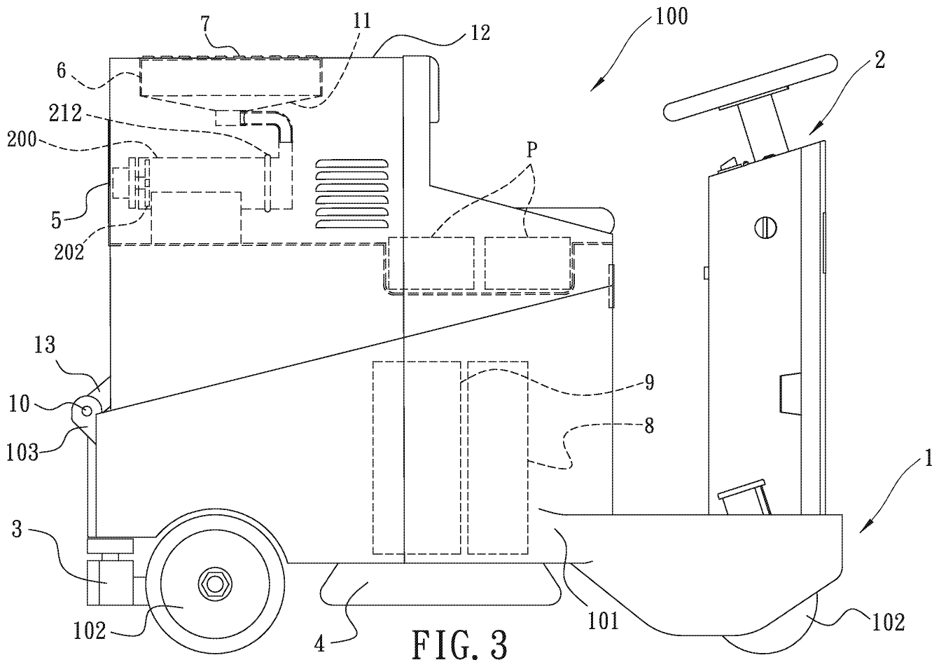

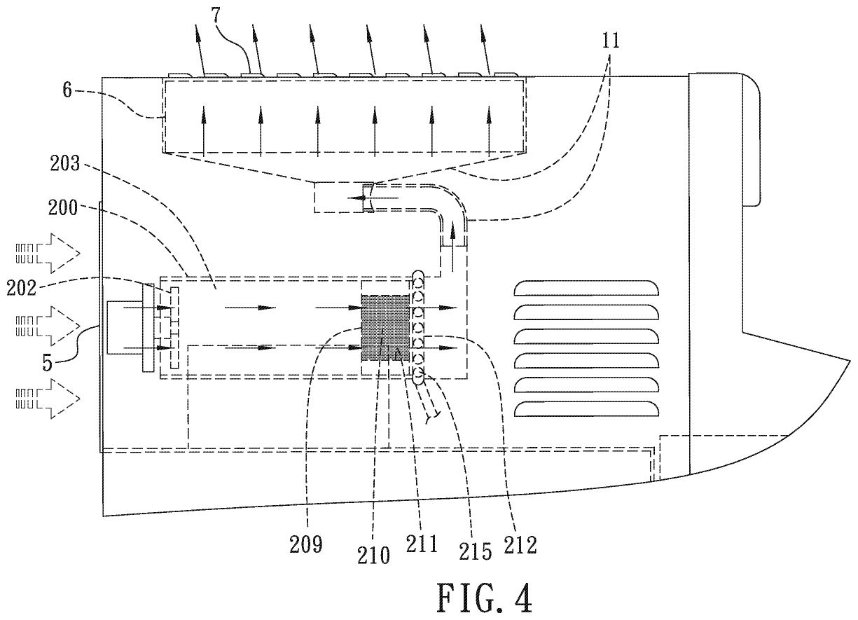

Referring to FIGS. 3 and 4, in operation, dirty airs flow into the sterilization structure 200 from the air inlet 5, in other words, the dirty airs in which bacteria and virus contain are drawn into the disinfecting tube 203 by the fan 202 and are discharged to the central opening 211. The heating ring 209 heats the stop mesh element 210 to 100° C. so that the stop mesh element 210 heats and stops the bacteria and the virus, thus obtaining disinfection. The multiple aerosol nozzles 215 of the rotatable spray ring 212 spray the carbon dioxide to sterilize the dirty airs, and the HEPA filter 6 filters the dirty airs, thus producing clean airs. Thereafter, the clean airs are discharged out of the air outlet 7 via the air conduit 11, thus eliminating the virus, such as COVID-19, SARS or Influenza.

While the first embodiments of the invention have been set forth for the purpose of disclosure, modifications of the disclosed embodiments of the invention as well as other embodiments thereof may occur to those skilled in the art. The scope of the claims should not be limited by the first embodiments set forth in the examples, but should be given the broadest interpretation consistent with the description as a whole.

What is claimed is:

1. A mobile disinfection cart comprising:
a body;
a drive system disposed on a front end of the body;
a water absorption rake fixed on a rear end of the body;
a brush disc mounted on a bottom of the body;
a lid arranged on a top of the rear end of the body;
an air inlet defined on a rear end of the lid and located above the rear end of the body;
a sterilization structure accommodated in the cover, located above the rear end of the chassis, facing the drive system, and communicating with the air inlet;
an air outlet defined on a top of the lid;
an air conduit communicating with the sterilization structure and the air outlet;
a filter fixed in an air conduit and located above the sterilization structure, and the air outlet being located above the filter;
a storage tank secured on the body and being in communication with the brush disc;
an accommodation box disposed on the body and being in communication with the sterilization structure; and
a power supply fixed on the body and electrically connected with the drive system and the sterilization structure.

2. The mobile disinfection cart as claimed in claim 1, wherein the sterilization structure includes a motor, a fan, a disinfecting tube, a main controller configured to control the motor and the fan, a heater controller, a delivery pipe configured to deliver chlorine dioxide, a control valve, a heating ring, a fixer configured to fix the heating ring, a stop mesh element, an opening, and a rotatable spray ring;
wherein the motor is electrically connected with the power supply, a front end of the fan is connected with the motor, and a rear end of the fan is coupled with the disinfecting tube, the main controller and the heater controller are mounted on an outer wall of the disinfecting tube and are electrically connected with the power supply; the delivery pipe is in communication with the accommodation box and the disinfecting tube, the control valve is fixed on a predetermined position of the delivery pipe and is controlled by the heater controller, the fixer is secured on an end of the disinfecting tube away from the fan, the heating ring is disposed on an end of the fixer away from the disinfecting tube, and the stop mesh element is mounted on an outer wall of an end of the heating ring away from the fixer, the central opening is defined on the end of the heating ring away from the fixer, and the central opening has a through hole formed on an inner wall thereof, the rotatable spray ring is fixed on an end of the central opening away from the heating ring.

3. The mobile disinfection cart as claimed in claim 1, wherein the filter is a High-Efficiency Particulate Air (HEPA) filter.

4. The mobile disinfection cart as claimed in claim 2, wherein the stop mesh element has 400 meshes.

5. The mobile disinfection cart as claimed in claim 2, wherein the rotatable spray ring has a coupling tube and multiple aerosol nozzles, the coupling tube passes through the through hole of the central opening and communicates with the accommodation box, and the multiple aerosol nozzles are spaced angularly, face inward, and communicate with the coupling tube.

6. The mobile disinfection cart as claimed in claim 1, wherein the storage tank and the accommodation box are defined between the drive system and the water absorption rake.

7. The mobile disinfection cart as claimed in claim 6, wherein the storage tank includes a detachable cap, and the accommodation box includes a removable cover.

8. The mobile disinfection cart as claimed in claim 1, wherein the chassis has a connection tab connected on the rear end of the lid by a rotary shaft so as to uncover and cover the lid.
